(19) United States
(12) Patent Application Publication
(10) Pub. No.: US 2019/0046309 A1
(43) Pub. Date: Feb. 14, 2019

(54) TOOTH PROCESSING DEVICE AND METHOD

(71) Applicants: BOE TECHNOLOGY GROUP CO., LTD., Beijing (CN); BEIJING BOE OPTOELECTRONICS TECHNOLOGY CO., LTD., Beijing (CN)

(72) Inventors: Yubing SONG, Beijing (CN); Yue GU, Beijing (CN); Mingjing WANG, Beijing (CN); Yi ZHENG, Beijing (CN); Qi SUN, Beijing (CN); Weipeng FU, Beijing (CN)

(73) Assignees: BOE TECHNOLOGY GROUP CO., LTD., Beijing (CN); BEIJING BOE OPTOELECTRONICS TECHNOLOGY CO., LTD., Beijing (CN)

(21) Appl. No.: 16/019,881

(22) Filed: Jun. 27, 2018

(30) Foreign Application Priority Data
Aug. 11, 2017 (CN) 201710687863.4

(51) Int. Cl. A61C 19/04; A61C 5/62; A61C 3/02; A61B 5/00

(52) U.S. Cl. CPC A61C 19/04; A61C 5/62; A61C 17/20; A61B 5/682; A61C 3/02

(57) ABSTRACT

The present disclosure relates to the field of medical equipment, and provides a tooth processing device and a tooth processing method. The tooth processing device includes: a dental model to be worn by a user and having a hollowed-out structure; an image collection circuit arranged on the dental model and configured to collect a tooth image; a dental drill arranged on the dental model and capable of moving on the dental model; and a processing circuit configured to analyze the collected tooth image, determine a decayed tooth portion, and control the dental drill to move to the decayed tooth portion so as to remove the decayed tooth portion through grinding.

### CROSS-REFERENCE TO RELATED APPLICATION

This application claims priority to Chinese Patent Application No. 201710687863.4 filed on Aug. 11, 2017, which is incorporated herein by reference in its entirety.

### TECHNICAL FIELD

The present disclosure relates to the field of medical equipment, in particular to a tooth processing device and a tooth processing method.

### BACKGROUND

Due to a bad dietary habit and an unhealthy living habit, people's teeth may be damaged at different levels, and a decayed tooth is the most common issue. In the case that the decayed tooth occurs, it is necessary to fill the decayed tooth. Usually, a dental filling operation is performed by a dentist. In the case that the dentist is of insufficient skills, the tooth may not be filled perfectly, and meanwhile there may exist a risk of inducing any other dental problems.

### SUMMARY

In one aspect, the present disclosure provides in some embodiments a tooth processing device, including: a dental model to be worn by a user and having a hollowed-out structure; an image collection circuit arranged on the dental model and configured to collect a tooth image; a dental drill arranged on the dental model and capable of moving on the dental model; and a processing circuit configured to analyze the collected tooth image, determine a decayed tooth portion, and control the dental drill to move to the decayed tooth portion so as to remove the decayed tooth portion through grinding.

In a possible embodiment of the present disclosure, the processing circuit includes a color comparison sub-circuit configured to compare a color of the tooth in the tooth image with a predetermined standard color, and determine a tooth portion whose color is different from the predetermined standard color as the decayed tooth portion.

In a possible embodiment of the present disclosure, the dental drill is provided with a receiver within which a dental filling material is received. The processing circuit is further configured to, after the decayed tooth portion has been completely removed, control the dental drill to inject the dental filling material to the ground tooth portion, so as to fill up a tooth missing portion.

In a possible embodiment of the present disclosure, the tooth processing device further includes a liquid supply port arranged in the dental model and in communication with an external liquid storage module. The processing circuit is further configured to, after the decayed tooth portion has been removed through grinding, inject a clean liquid into a user's mouth via the liquid supply port.

In a possible embodiment of the present disclosure, the tooth processing device further includes an ultrasonic wave generation circuit arranged on the dental model. The processing circuit is further configured to, after the clean liquid has been injected into the user's mouth via the liquid supply port, control the ultrasonic wave generation circuit to generate an ultrasonic wave so as to vibrate the clean liquid.

In a possible embodiment of the present disclosure, the tooth processing device further includes a liquid discharge port arranged in the dental model. The processing circuit is further configured to, after the clean liquid has been injected into the user's mouth via the liquid supply port, discharge a waste liquid in the user's mouth via the liquid discharge port.

In a possible embodiment of the present disclosure, the tooth processing device further includes an infrared scanning circuit. The processing circuit is further configured to, after the clean liquid has been injected into the user's mouth via the liquid supply port, control the infrared scanning circuit to scan the user's mouth, and determine a position and a shape of the tooth missing portion in accordance with a boundary between the clean liquid and the tooth in an infrared scanning image acquired by the infrared scanning circuit.

In a possible embodiment of the present disclosure, the dental model is made of a deformable material. The processing circuit is further configured to, after the tooth occlusion, determine the position and the shape of the tooth missing portion in accordance with a deformation amount of the dental model.

In a possible embodiment of the present disclosure, the tooth processing device further includes a housing covering an outer surface of the dental model and provided at an inner surface with bristles.

In a possible embodiment of the present disclosure, a guide rail is arranged on the dental model, a sliding block is arranged on the guide rail, and the dental drill is fixed onto the sliding block. The processing circuit is further configured to control the movement of the sliding block, so as to control the dental drill to move on the dental model to the decayed tooth portion.

In a possible embodiment of the present disclosure, the processing circuit is further connected to an analyzation device arranged outside the tooth processing device, and configured to transmit the deformation amount of the dental model to the analyzation device. The analyzation device is configured to determine a tooth surface repairing level.

In a possible embodiment of the present disclosure, the analyzation device is a computer.

In a possible embodiment of the present disclosure, the processing circuit is connected to the analyzation device in a wireless manner.

In a possible embodiment of the present disclosure, the processing circuit is connected to the analyzation device in a wired manner.

In another aspect, the present disclosure provides in some embodiments a tooth processing method for use in the above-mentioned tooth processing device, including steps of: after the dental model is worn by a user, collecting, by the image collection circuit, a tooth image; and analyzing, by the processing circuit, the collected tooth image so as to determine a decayed tooth portion, and controlling the dental drill to move to the decayed tooth portion so as to remove the decayed tooth portion through grinding.

In a possible embodiment of the present disclosure, the dental drill is provided with a receiver within which a dental filling material is received. The tooth processing method further includes, after the decayed tooth portion has been completely removed, controlling, by the processing circuit, the dental drill to inject the dental filling material to the ground tooth portion, so as to fill up a tooth missing portion.

In a possible embodiment of the present disclosure, the tooth processing method further includes enabling the dental model to cover the user's tooth in the case that dental model is worn by the user initially, enabling, by the processing circuit, the dental model to be expanded in the case that the decayed tooth portion needs to be removed through grinding so as to expose the user's tooth, and thereby controlling the dental drill to be in contact with the user's tooth so as to remove the decayed tooth portion through grinding.

In a possible embodiment of the present disclosure, the tooth processing method further includes, during the removable of the decayed tooth portion through grinding, collecting, by the image collection circuit, the tooth image continuously, so as to compare the tooth image with a predetermined standard image in real time.

In a possible embodiment of the present disclosure, the dental drill is provided with a receiver within which a dental filling material is received. The tooth processing method further includes, after the decayed tooth portion has been completely removed, controlling, by the processing circuit, the dental drill to inject the dental filling material to the ground tooth portion, so as to fill up a tooth missing portion.

In a possible embodiment of the present disclosure, the tooth processing device further includes a liquid supply port arranged in the dental model and in communication with an external liquid storage module. The tooth processing method further includes, after the decayed tooth portion has been removed through grinding, injecting a clean liquid into a user's mouth via the liquid supply port.

### BRIEF DESCRIPTION OF THE DRAWINGS

FIG. 1 is a schematic view showing a tooth processing device according to one embodiment of the present disclosure;

FIG. 2 is another schematic view showing the tooth processing device according to another embodiment of the present disclosure; and

FIG. 3 is a schematic view showing the movement of a dental drill along a guide rail according to embodiments of the present disclosure.

### REFERENCE SIGN LIST

1 dental model
2 processing circuit
3 dental drill
4 guide rail
5 liquid supply port
6 liquid discharge port
7 housing
8 bristles
9 analyzation device
10 sliding block

### DETAILED DESCRIPTION

The present disclosure will be described hereinafter in conjunction with the drawings and embodiments. The following embodiments are for illustrative purposes only, but shall not be used to limit the scope of the present disclosure.

An object of the present disclosure is to provide a tooth processing device and a tooth processing method, so as to remove a decayed tooth portion of a user accurately.

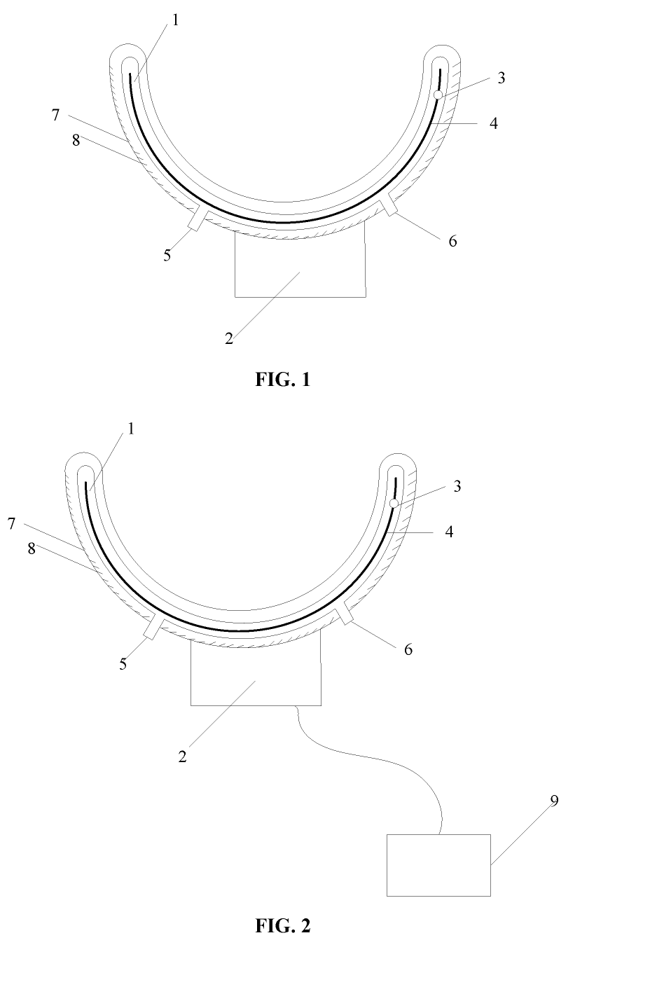

The present disclosure provides in some embodiments a tooth processing device which, as shown in FIG. 1 and FIG. 2, includes: a dental model 1 to be worn by a user and having a hollowed-out structure; an image collection circuit (not shown) arranged on the dental model 1 and configured to collect a tooth image; a dental drill 3 arranged on the dental model 1 and capable of moving on the dental model 1; and a processing circuit 2 configured to analyze the collected tooth image, determine a decayed tooth portion, and control the dental drill 3 to move to the decayed tooth portion so as to remove the decayed tooth portion through grinding.

According to the tooth processing device in the embodiments of the present disclosure, in the case that it is necessary to remove the decayed tooth portion, the tooth image of the user is collected by the image collection circuit on the dental model 1 worn by the user, then the collected tooth image is analyzed to determine the decayed tooth portion, and then the dental drill 3 is moved to the decayed tooth portion, so as to remove the decayed tooth portion through grinding. As a result, it is able to remove the decayed tooth portion accurately through image analysis, independent of skills of a dentist.

The dental model 1 is of a hollowed-out housing having a shape approximately identical to teeth of the user. In the case that tooth problems occur and the dental model 1 is worn by the user, the tooth image may be taken by the image collection circuit on the dental model 1. To be specific, the dental model 1 includes an upper portion and a lower portion. In the case that the dental model 1 is worn by the user, the upper portion of the dental model 1 corresponds to upper teeth of the user, while the lower portion corresponds to lower teeth of the user. The image collection circuit may be arranged at a side of the upper portion to collect an image of the lower teeth, and arranged a side of the lower portion to collect an image of the upper teeth.

The decayed tooth portion is black while a normal tooth portion is white or light yellow, so it is able to determine the decayed tooth portion through color comparison. To be specific, the processing circuit 2 includes a color comparison sub-circuit (not shown) configured to compare a color of the tooth in the tooth image with a predetermined standard color, and determine a tooth portion whose color is different from the predetermined standard color as the decayed tooth portion. Then, the dental drill 3 may be moved to the decayed tooth portion under the control of the processing circuit 2, so as to remove the decayed tooth portion through grinding.

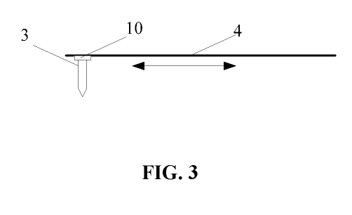

The dental drill 3 is capable of moving freely on the dental model 1. To be specific, as shown in FIG. 3, a guide rail 4 is arranged on the dental model 1, a sliding block 10 is arranged on the guide rail 4, and the dental drill 3 is fixed onto the sliding block 10. A control module of the processing circuit 2 is configured to control the movement of the sliding block 10, so as to move the dental drill 3 in a horizontal direction on the dental model 1, thereby to move the dental drill 3 to the decayed tooth portion. In the case that the dental model 1 is worn by the user initially, the dental model 1 may cover the user's teeth. In the case that it is necessary to remove the decayed tooth portion through grinding, the control module may control the dental model 1 to be expanded, so as to expose the user's teeth, thereby to control the dental drill 3 to be in contact with the decayed tooth portion and remove the decayed tooth portion through grinding. During the removal, the image collection circuit may collect the tooth image continuously, and compare it with the predetermined standard image in real time. In this way, it is able to completely remove the decayed tooth portion through the dental drill 3, without damaging a healthy portion of the tooth.

In addition, as shown in FIG. 1 and FIG. 2, the tooth processing device may further include a liquid supply port 5 arranged in the dental model 1 and in communication with an external liquid storage module. The processing circuit 2 is further configured to, after the decayed tooth portion has been removed through grinding, inject a clean liquid into a user's mouth via the liquid supply port 5.

After the removal of the decayed tooth portion through grinding, the clean liquid may be injected into the user's mouth, so as to clean the user's mouth. To be specific, the clean liquid may be water.

In addition, the tooth processing device may further include an ultrasonic wave generation circuit (not shown) arranged on the dental model 1. The processing circuit 2 is further configured to, after the clean liquid has been injected into the user's mouth via the liquid supply port 5, control the ultrasonic wave generation circuit to generate an ultrasonic wave so as to vibrate the clean liquid. Through the ultrasonic wave, it is able to clean a surface of the tooth and a slit between the teeth in a better manner.

In addition, as shown in FIG. 1 and FIG. 2, the tooth processing device may further include a liquid discharge port 6 arranged in the dental model 1. The processing circuit 2 is further configured to, after the clean liquid has been injected into the user's mouth via the liquid supply port 5, discharge a waste liquid in the user's mouth via the liquid discharge port 6. The clean liquid is injected into the user's mouth via the liquid supply port 5, so it is necessary to discharge the waste liquid in the user's mouth via the liquid discharge port 6.

Here, as shown in FIG. 1 and FIG. 2, the liquid supply port 5 and the liquid discharge port 6 may be arranged symmetrically relative to the processing circuit 2. It should be appreciated that, through the liquid supply port 5, the ultrasonic wave generation circuit and the liquid discharge port 6, it is able to clean the user's mouth in a better manner.

In addition, after the removal of the decayed tooth portion, it is also necessary to fill up the tooth missing portion. Prior to filling up the tooth missing portion, it is necessary to determine an amount of a dental filling material to be used.

In a possible embodiment of the present disclosure, the tooth processing device further includes an infrared scanning circuit (not shown). After the injection of the clean liquid into the user's mouth via the liquid supply port 5, the infrared scanning circuit is configured to scan the user's mouth, and determine a position and a shape of the tooth missing portion in accordance with a boundary between the clean liquid and the tooth in an infrared scanning image acquired by the infrared scanning circuit, so as to determine the amount of the dental filling material to be used.

In another possible embodiment of the present disclosure, the dental model 1 is made of a deformable material, which may be selected in accordance with the practical need. The processing circuit 2 is further configured to, after the tooth occlusion, determine the position and the shape of the tooth missing portion in accordance with a deformation amount of the dental model 1, so as to determine the amount of the dental filling material to be used.

In addition, as shown in FIG. 2, the processing circuit 2 may be further connected to an external analyzation device 9, e.g., a computer. The processing circuit 2 is further configured to transmit the deformation amount of the dental model 1 to the analyzation device 9. The analyzation device 9 determines a repair level of a filled surface of the tooth to match an opposite surface of the tooth, so as to determine the amount of the dental filling material to be used. Here, the processing circuit 2 may be connected to the external analyzation device 9, e.g., the computer, in a wireless or wired manner.

After the determination of the amount of the dental filling material to be used, the dental filling material may be injected to the ground tooth portion via the dental drill 3. The dental drill 3 is provided with a receiver for receiving therein the dental filling material. The processing circuit 2 is further configured to, after the decayed tooth portion has been completely removed, inject the dental filling material to the ground tooth portion via the dental drill 3, so as to fill up the tooth missing portion. In this way, it is able to fill up the tooth missing portion through the dental drill 3, rather than using any additional device. The dental filling material may be injected into the receiver in the dental drill 3 via the liquid supply port 5, and injected to the tooth missing portion via a hole in the dental drill 3. Due to its ultra-thin structure, it is able for the dental drill 3 to fill up the tooth missing portion to the maximum extent. As a result, it is able to prevent the occurrence of any other tooth problems due to the incomplete dental filling operation, and meanwhile facilitate the removal of impurities as compared with the related art where an oversize slit occurs in the case that the dental filling operation is performed manually.

In addition, as shown in FIG. 1 and FIG. 2, the tooth processing device may further include a housing 7 covering an outer surface of the dental model 1 and provided at an inner surface with bristles 8. In this way, the tooth processing device is further provided with a tooth cleaning function. In the case that the function is enabled, the dental model 1 may not cover the user's tooth any more under the control of the processing circuit 2, and the bristles 8 between the model 1 and the housing 7 may be in contact with the teeth. Due to an ultra-thin structure, the bristles 8 may easily extend into the slits between the teeth, so as to clean the teeth completely.

The present disclosure further provides in some embodiments a tooth processing method for use in the above-mentioned tooth processing device, which includes steps of: after the dental model 1 is worn by the user, collecting, by the image collection circuit, the tooth image; and analyzing, by the processing circuit 2, the collected tooth image so as to determine the decayed tooth portion, and controlling the dental drill 3 to move to the decayed tooth portion so as to remove the decayed tooth portion through grinding.

According to the tooth processing method in the embodiments of the present disclosure, in the case that it is necessary to remove the decayed tooth portion, the tooth image of the user is collected by the image collection circuit on the dental model 1 worn by the user, then the collected tooth image is analyzed to determine the decayed tooth portion, and then the dental drill 3 is moved to the decayed tooth portion, so as to remove the decayed tooth portion through grinding. As a result, it is able to remove the decayed tooth portion accurately through image analysis, independent of skills of a dentist.

The decayed tooth portion is black while a normal tooth portion is white or light yellow, so it is able to determine the decayed tooth portion through color comparison.

In addition, after the removal of the decayed tooth portion, it is also necessary to fill up the tooth missing portion. Prior to filling up the tooth missing portion, it is necessary to determine an amount of a dental filling material to be used.

In a possible embodiment of the present disclosure, the tooth processing device further includes an infrared scanning circuit (not shown). After the injection of the clean liquid into the user's mouth via the liquid supply port 5, the infrared scanning circuit is configured to scan the user's mouth, and determine a position and a shape of the tooth missing portion in accordance with a boundary between the clean liquid and the tooth in an infrared scanning image acquired by the infrared scanning circuit, so as to determine the amount of the dental filling material to be used.

In another possible embodiment of the present disclosure, the dental model 1 is made of a deformable material, which may be selected in accordance with the practical need. The processing circuit 2 is further configured to, after the tooth occlusion, determine the position and the shape of the tooth missing portion in accordance with a deformation amount of the dental model 1, so as to determine the amount of the dental filling material to be used.

In addition, as shown in FIG. 2, the processing circuit 2 may be further connected to an external analyzation device 9, e.g., a computer. The processing circuit 2 is further configured to transmit the deformation amount of the dental model 1 to the analyzation device 9. The analyzation device 9 determines a repair level of a filled surface of the tooth to match an opposite surface of the tooth, so as to determine the amount of the dental filling material to be used. Here, the processing circuit 2 may be connected to the external analyzation device 9, e.g., the computer, in a wireless or wired manner.

After the determination of the amount of the dental filling material to be used, the dental filling material may be injected to the ground tooth portion via the dental drill 3. The dental drill 3 is provided with a receiver for receiving therein the dental filling material. The processing circuit 2 is further configured to, after the decayed tooth portion has been completely removed, inject the dental filling material to the ground tooth portion via the dental drill 3, so as to fill up the tooth missing portion. In this way, it is able to fill up the tooth missing portion through the dental drill 3, rather than using any additional device. The dental filling material may be injected into the receiver in the dental drill 3 via the liquid supply port 5, and injected to the tooth missing portion via a hole in the dental drill 3. Due to its ultra-thin structure, it is able for the dental drill 3 to fill up the tooth missing part to the maximum extent. As a result, it is able to prevent the occurrence of any other tooth problems due to the incomplete dental filling operation, and meanwhile facilitate the removal of impurities as compared with the related art where an oversize slit occurs in the case that the dental filling operation is performed manually.

Unless otherwise defined, any technical or scientific term used herein shall have the common meaning understood by a person of ordinary skills. Such words as “first” and “second” used in the specification and claims are merely used to differentiate different components rather than to represent any order, number or importance. Similarly, such words as “one” or “one of” are merely used to represent the existence of at least one member, rather than to limit the number thereof. Such words as “connect” or “connected to” may include electrical connection, direct or indirect, rather than to be limited to physical or mechanical connection. Such words as “on”, “under”, “left” and “right” are merely used to represent relative position relationship, and when an absolute position of the object is changed, the relative position relationship will be changed too.

It should be appreciated that, in the case that such an element as layer, film, region or substrate is arranged “on” or “under” another element, it may be directly arranged “on” or “under” the other substrate, or an intermediate element may be arranged therebetween.

A person skilled in the art may make further modifications and improvements without departing from the spirit of the present disclosure, and these modifications and improvements shall also fall within the scope of the present disclosure.

What is claimed is:

1. A tooth processing device, comprising:
a dental model to be worn by a user and having a hollowed-out structure;
an image collection circuit arranged on the dental model and configured to collect a tooth image;
a dental drill arranged on the dental model and capable of moving on the dental model; and
a processing circuit configured to analyze the collected tooth image, determine a decayed tooth portion, and control the dental drill to move to the decayed tooth portion so as to remove the decayed tooth portion through grinding.

2. The tooth processing device according to claim 1, wherein the processing circuit comprises a color comparison sub-circuit configured to compare a color of the tooth in the tooth image with a predetermined standard color, and determine a tooth portion whose color is different from the predetermined standard color as the decayed tooth portion.

3. The tooth processing device according to claim 1, wherein the dental drill is provided with a receiver within which a dental filling material is received, and the processing circuit is further configured to, after the decayed tooth portion has been completely removed, control the dental drill to inject the dental filling material to the ground tooth portion, so as to fill up a tooth missing portion.

4. The tooth processing device according to claim 3, further comprising a liquid supply port arranged in the dental model and in communication with an external liquid storage module, wherein the processing circuit is further configured to, after the decayed tooth portion has been removed through grinding, inject a clean liquid into a user's mouth via the liquid supply port.

5. The tooth processing device according to claim 4, further comprising an ultrasonic wave generation circuit arranged on the dental model, wherein the processing circuit is further configured to, after the clean liquid has been injected into the user's mouth via the liquid supply port, control the ultrasonic wave generation circuit to generate an ultrasonic wave so as to vibrate the clean liquid.

6. The tooth processing device according to claim 4, further comprising a liquid discharge port arranged in the dental model, wherein the processing circuit is further configured to, after the clean liquid has been injected into the user's mouth via the liquid supply port, discharge a waste liquid in the user's mouth via the liquid discharge port.

7. The tooth processing device according to claim 4, further comprising an infrared scanning circuit, wherein the processing circuit is further configured to, after the clean liquid has been injected into the user's mouth via the liquid supply port, control the infrared scanning circuit to scan the user's mouth, and determine a position and a shape of the tooth missing portion in accordance with a boundary between the clean liquid and the tooth in an infrared scanning image acquired by the infrared scanning circuit.

8. The tooth processing device according to claim 2, wherein the dental model is made of a deformable material, and the processing circuit is further configured to, after the tooth occlusion, determine the position and the shape of the tooth missing portion in accordance with a deformation amount of the dental model.

9. The tooth processing device according to claim 1, further comprising a housing covering an outer surface of the dental model and provided at an inner surface with bristles.

10. The tooth processing device according to claim 1, wherein a guide rail is arranged on the dental model, a sliding block is arranged on the guide rail, and the dental drill is fixed onto the sliding block, wherein the processing circuit is further configured to control the movement of the sliding block, so as to control the dental drill to move on the dental model to the decayed tooth portion.

11. The tooth processing device according to claim 1, wherein the processing circuit is further connected to an analyzation device arranged outside the tooth processing device, and configured to transmit the deformation amount of the dental model to the analyzation device, wherein the analyzation device is configured to determine a tooth surface repairing level.

12. The tooth processing device according to claim 11, wherein the analyzation device is a computer.

13. The tooth processing device according to claim 11, wherein the processing circuit is connected to the analyzation device in a wireless manner.

14. The tooth processing device according to claim 11, wherein the processing circuit is connected to the analyzation device in a wired manner.

15. A tooth processing method for use in a tooth processing device, wherein the tooth processing device comprises a dental model to be worn by a user and having a hollowed-out structure, an image collection circuit arranged on the dental model and configured to collect a tooth image, a dental drill arranged on the dental model and capable of moving on the dental model, and a processing circuit configured to analyze the collected tooth image, determine a decayed tooth portion, and control the dental drill to move to the decayed tooth portion so as to remove the decayed tooth portion through grinding,
the tooth processing method comprising:
collecting, by the image collection circuit, the tooth image, after the dental model is worn by the user; and
analyzing, by the processing circuit, the collected tooth image so as to determine the decayed tooth portion, and controlling the dental drill to move to the decayed tooth portion so as to remove the decayed tooth portion through grinding.

16. The tooth processing method according to claim 15, wherein the dental drill is provided with a receiver within which a dental filling material is received, and the tooth processing method further comprises: controlling, by the processing circuit, the dental drill to inject the dental filling material to the ground tooth portion, so as to fill up a tooth missing portion, after the decayed tooth portion has been completely removed.

17. The tooth processing method according to claim 15, further comprising: enabling the dental model to cover the user's tooth in the case that dental model is worn by the user initially, enabling, by the processing circuit, the dental model to be expanded in the case that the decayed tooth portion needs to be removed through grinding so as to expose the user's tooth, and thereby controlling the dental drill to be in contact with the user's tooth so as to remove the decayed tooth portion through grinding.

18. The tooth processing method according to claim 15, further comprising: collecting, by the image collection circuit, the tooth image continuously, so as to compare the tooth image with a predetermined standard image in real time, during the removable of the decayed tooth portion through grinding.

19. The tooth processing method according to claim 15, wherein the dental drill is provided with a receiver within which a dental filling material is received, and the tooth processing method further comprises: controlling, by the processing circuit, the dental drill to inject the dental filling material to the ground tooth portion, so as to fill up a tooth missing portion, after the decayed tooth portion has been completely removed.

20. The tooth processing method according to claim 19, wherein the tooth processing device further comprises a liquid supply port arranged in the dental model and in communication with an external liquid storage module, and the tooth processing method further comprises: injecting a clean liquid into a user's mouth via the liquid supply port, after the decayed tooth portion has been removed through grinding.
